(19) United States
(12) Patent Application Publication
(10) Pub. No.: US 2014/0363373 A1
(43) Pub. Date: Dec. 11, 2014

(54) METHOD FOR COMBINED IMAGING AND TREATING ORGANS AND TISSUES

(71) Applicant: ONCOFLUOR, INC., Coronado, CA (US)

(72) Inventor: George A. Luiken, Coronado, CA (US)

(21) Appl. No.: 14/468,151

(22) Filed: Aug. 25, 2014

Related U.S. Application Data
(63) Continuation of application No. 13/358,380, filed on Jan. 25, 2012.
(60) Provisional application No. 61/436,133, filed on Jan. 25, 2011.

(51) Int. Cl. A61K 49/00; A61K 47/48; A61K 51/10

(52) U.S. Cl. CPC A61K 49/0058; A61K 51/1045; A61K 51/1066; A61K 51/1051; A61K 51/1054; A61K 51/1072; A61K 51/1063; A61K 51/106; A61K 47/484
USPC 424/1.49; 424/178.1

(57) ABSTRACT

The present invention provides methods and compositions for detecting and treating malignant tissue, organs or cells in a mammal. The method comprises parenterally injecting a mammalian subject, at a locus or by a route providing access to the tissue or organ, with a composition comprising a monoclonal antibody (chimeric, humanized, fully human), partial antibody, Fab Fragment, antibody fragment that is tagged with a fluorophore with or without the addition of a therapeutic chemotherapy molecule, which specifically binds to the targeted organ, tissue or cell. Resection of the primary malignant tissue within the mammalian species (using the fluorescence of the fluorescing targeting construct) provides the advantage of identifying all bulk tumor as fluorescent at the time of the original tumor resection. Additional (adjuvant) therapy is provided by the chemotherapy molecule that is bound to the fluorescent-tagged monoclonal antibody (or antibody part thereof that is bound to small, microscopic clusters of cells (circulating tumor cells or tissue bound small microscopic clusters of cells) that are not visible to the naked eye and that could not be seen with the aid of the excitation light source and a magnification device. Chemotherapy molecules bound to the fluorescent-tagged monoclonal antibody construct provide the additional benefit of killing off the malignant cells that might be undetected using just the excitation light source for surgical resection.

### CROSS REFERENCE TO RELATED APPLICATIONS

This application claims the benefit of priority under 35 U.S.C. §119(e) of U.S. Ser. No. 61/436,133, filed Jan. 25, 2011, the entire contents of which are incorporated herein by reference in its entirety.

### BACKGROUND OF THE INVENTION

### Field of the Invention

The present invention relates to methods for viewing the state of a body cavity or an internal organ of a mammalian body to allow more accurate removal of diseased tissue, and more particularly, to methods for detecting tumor tissue at an interior body site using a fluorescent targeting construct excited by light in the visible light range, and to treating such tissues, in an adjuvant and/or primary treatment manner.

Many solid and liquid substances naturally emit fluorescent radiation when irradiated or illuminated with ultraviolet (UV), visible, or near-infrared (NIR) light. However, the radiation may fall within wide wavelength bands of low intensity. In the case of many natural objects, observations are partially obscured by natural fluorescence emanating simultaneously from many different compounds present in the sample under examination. In imaging devices such as microscopes and charged couple devices (CCDs), therefore, it is known to employ a filter for a selected wavelength band to screen out undesired fluorescence emanating from the object under observation in order to view the desired area of fluorescence.

In medical applications, a similar difficulty arises because both tumors and healthy tissue fluoresce naturally (auto fluorescence), albeit often at different wavelengths. Consequently, when light-activated (UV, visible or NIR) fluorescence is used to detect tumors against a background of healthy tissue, identification of tumor tissue may be difficult. However, unlike most other cells of the body, tumor cells may possess a natural ability to concentrate and retain hematoporphyrin derivative dyes. Based upon this discovery, a technique was developed wherein a hematoporphyrin derivative fluorescent dye is administered and allowed to concentrate in a tumor to be examined to increase the fluorescence from the tumor as compared with that of healthy background tissue. Hematoporphyrin dyes fluoresce within a fluorescence spectrum between 610 and 700 nm, a spectrum easy to detect. However, the natural fluorescence from healthy cells may be much more intense than that from the dyes, and has a broader fluorescence spectrum. Thus, the use of fluorescent dyes in diagnosis of tumors has not been wholly successful.

In endoscopic systems, it is also known to irradiate an internal organ with visible radiation to obtain a visible image and then to apply to the internal organ a fluorescent dye that concentrates in tumors over a period of time. The dye is allowed to concentrate, and then the internal organ is irradiated with excitation radiation specific the dye to obtain a second fluorescent image. A body part having abnormal or diseased tissue, such as a cancer, may be identified by comparing an image produced by visible radiation of the internal organ with the image produced by fluorescence. To aid in visualizing the images received, endoscopic systems commonly utilize a still or video camera attached to a fiber optic scope having an optical guide fiber for guiding a beam from an external radiation source to the internal organ, and another optical guide fiber for transmitting a fluorescent image of the affected area to a monitor for viewing. These two approaches are combined in a method of the type disclosed in U.S. Pat. No. 4,821,117, wherein a fluorescent dye is applied to an object to be inspected, is allowed to concentrate in the tumor, and the affected site is then alternately irradiated with visible light and with radiation at the excitation wavelength of the fluorophore. Images of the object obtained independently by visible and fluorescent light using a TV camera are stored in memory, and are simultaneously displayed in a television monitor to visually distinguish the affected area of the body part from the healthy background tissue.

In another type of procedure, such as is described in U.S. Pat. No. 4,786,813, a beam-splitting system splits the fluorescence radiation passing though the optical system into at least three parts, each of which forms a respective image of the object corresponding to each of the wavelength regions received. A detector produces a cumulative weighted signal for each image point corresponding to a single point on the object. From the weighted signal values of the various points on the object, an image of the object having improved contrast is produced. This technique is used to aid in distinguishing the fluorescence from the affected tissue from that produced by normal tissue.

A still more complex method of visualizing images from an endoscopic device uses a television scanning apparatus. For example, U.S. Pat. No. 4,719,508 discloses a method utilizing an endoscopic photographing apparatus wherein the endoscope includes an image sensor for successively generating image signals fed to a first frame memory for storing the image signals and a second frame memory for interlacing and storing image signals read successively from the first frame memory. The stored, interlaced image signals are delivered to a TV monitor for display to aid in visualizing the affected body part.

These prior art endoscopic systems, which rely on photographic processing of the image of the area of interest (i.e., via a TV monitor), while effective, have historically relied on increasingly complex and expensive equipment and substitute image processing to construct a diagnostic image (i.e., indirect viewing) for direct viewing of the affected body part without image processing, as by any type of camera or image processing device. A major shortfall of these prior art systems is that they all require specialized operator training and expertise, expensive, complex and technically sophisticated equipment, and may not be generally available in community medical facilities. In addition, these prior art systems may increase the time required to complete a surgical procedure, thereby adding to the patient's time under anesthesia, and subsequent risks therefrom. Finally, if the technology fails at any time during the operative procedure, there is no advantage over direct visualization.

Certain of the fluorescent dyes that concentrate in tumors due to natural bodily processes can be excited at wavelengths corresponding to those produced by lasers to accomplish diagnostic and therapeutic purposes. Consequently, lasers have also been used in procedures utilizing endoscopic systems in conjunction with fluorescent dyes to image and treat tumors. In one embodiment of this general method, a dye is used that absorbs laser light at two different wavelengths and/or laser powers, one that excites fluorescence without generating damaging heat in the tissue, and one that generates sufficient heat in the dye to destroy surrounding tissue. U.S. Pat. No. 4,768,513, for example, discloses a procedure in which a dye is applied to a body part suspected of containing a tumor, usually by local injection. The dye is allowed to concentrate in tumors and clear from healthy tissue over a period of days, and then the body part is irradiated with alternate pulses of two light sources: a white light of a known intensity and a fluorescence exciting laser light. To compensate for variations in intensity of the fluorescence resulting from variations in the angle of incident light, and the like, visualization of the tumor is computer enhanced by calculating the intensity of the fluorescence with respect to the known intensity of the white light. Ablation of a tumor detected using this method is accomplished by switching the laser to the heat-generating wavelength so as to destroy the cancerous tissue into which the fluorophore has collected.

While effective for diagnosing and treating tumors, such methods have two major drawbacks. Disease states other than tumors cannot be diagnosed, and laser visualization must be delayed for a period of two days or more after administration of the fluorescent dye to allow the dye to clear from normal tissue.

Monoclonal antibodies and other tumor-avid compounds specific for tumors have been developed for use in diagnosis of tumors, both in tissue samples and in vivo. In addition to such ligands, certain tumor-avid moieties are disproportionately taken up (and, or optionally are metabolized by tumor cells). Several well-known tumor-avid compounds are deoxyglucose, which plays a role in glycolysis in tumor cells; somatostatin, which binds to and/or is taken up by somatostatin receptors in tumor cells and particularly in endocrine tumors; and methionine, histidine and folic acid, which are used as a substrate for metabolism in a wide array of tissues.

In such studies, deoxyglucose is used as a radio-tagged moiety, such as fluorodeoxyglucose (¹⁸F-deoxyglucose), for detection of tumors of various types. It is believed that tumor cells experience such a mismatch between glucose consumption and glucose delivery that anaerobic glycolysis must be relied upon, thereby elevating the concentration of the radioactive tag in tumor tissue. It is also a possibility that the elevated concentration of deoxyglucose in malignant tumors may be caused by the presence of isoenzymes of hexokinase with abnormal affinities for native glucose or its analogs (A. Gjedde, Chapter 6: “Glucose Metabolism,” Principles of Nuclear Medicine, 2nd Ed., W.B. Saunders Company, Philadelphia, Pa., pages 54-69). Similarly, due to the concentration of methionine and somatostatin in tumor tissue, radio-tagged methionine and somatostatin, and fragments or analogs thereof, are used in the art for non-invasive imaging of a variety of tumor types. One such procedure is known as somatostatin receptor scintigraphy (SRS).

Although these techniques have met with considerable success in determining the presence of tumor tissue, scintigraphic techniques are difficult to apply during a surgical procedure because of the equipment necessary for viewing the image provided by the radioisotope. Yet it is exactly at the time that the surgeon has made the incision or entered the body cavity that it would be most useful to “see” the outlines of the diseased tissue in real time and without the need for time-consuming, expensive image processing equipment. In addition, even using the best surgical techniques, it is well known that residual microscopic clusters of cells can and frequently are left behind after surgical excision of malignant tissue.

Thus, there is a need in the art for improved methods that can be used to directly visualize a broad range of putative disease sites without the need for use of image processing equipment as well as eliminate microscopic residual disease cells or clusters which are not typically visible to the naked eye, but which can lead to local or distant recurrence of a malignancy. Where real-time visualization is by means of endoscopic devices, direct visualization (as opposed to images created by image processing equipment) offers the additional advantage that the equipment required is comparatively simple to use, is not prone to malfunction, and is less expensive than the equipment required to process images or create photographic displays from such images and no additional time is spent in image processing. In addition, there is a need in the art for a method of identifying diseased or abnormal tissue during surgical procedures so that immediate resection or biopsy of the identified tissue can be performed while the surgeon “sees” the outlines of the diseased or abnormal tissue.

The use of adjuvant chemotherapy (adjunctive or additional chemotherapy given following primary surgery for cancer) to improve survival following surgery is well established. The utility of adjuvant chemotherapy is due to the ability to kill cancer cells that are not removed at the time of surgery and that may have spread from the primary tumor prior to removal of the tumor. The benefit of adjuvant chemotherapy has been demonstrated most consistently for patients with breast cancer, lung, colon and testicular cancer and is being used more frequently in other tumors as well. Adjuvant chemotherapy is typically given for several weeks to months following the initial surgical resection.

Direct delivery of chemotherapy drugs to the tumor tissue can be obtained by linking the drugs to tumor-specific MAbs or tumor-avid compounds. In order for this concept to work, it is important to know, prior to the use of these compounds, that they bind selectively to tumor tissue and only minimally or not at all to normal tissue. The expression of tumor antigens by malignant cells provides one means of selective delivery of the MAb bound chemotherapy drugs. Linking a therapeutic drug (i.e. chemotherapy, hormone, small tumor-targeted molecule, etc) to a fluorescence-tagged tumor-specific construct (MAb or tumor-avid compound), would offer the chance to accurately identify and surgically remove all visible tumor tissue and to destroy microscopic tumor cell clusters through the direct cellular delivery of therapeutic drug bound to the MAb. This direct initial treatment (surgical removal) coupled with the adjuvant treatment (therapeutic drug bound to tumor specific MAb) offers the potential to improve cure rates for a wide variety of malignancies without the patient having to undergo systemic chemotherapy after the initial surgical intervention. The addition of the therapeutic drug delivery would in essence provide “adjuvant” therapy to kill any small clusters of tumor cells that would not typically be visible using the imaging techniques described.

### SUMMARY OF THE INVENTION

The present invention overcomes many of these problems in the art by providing method(s) for in vivo identification of diseased tissue in a subject in need thereof. As such, the present invention relates to methods for visually detecting tumor tissue at an interior or exterior body site using tumor-specific fluorescent targeting constructs, which are excited by light in the visible range (i.e. 401-510 nm), to allow more accurate removal of all diseased tissue, and for treating residual microscopic or macroscopic tumor tissue with therapeutic drug (chemotherapy, hormone, etc) attached to the tumor-specific fluorescent targeting constructs.

The invention method includes illuminating an in vivo body part of the subject containing diseased tissue with light having at least one excitation wavelength in the range from about 401 nm to about 510 nm. Fluorescence emanating from a fluorescent targeting construct administered to the subject and which has specifically bound to and/or been taken up by the diseased tissue in the body part, in response to the at least one excitation wavelength is directly viewed to determine the location and/or surface area of the diseased tissue in the subject.

In one embodiment, the fluorescent targeting construct comprises a fluorophore-tagged antibody (partial antibody, Fab fragment, diabody) or fluorophore-tagged tumor avid moiety and a therapeutic drug (chemotherapy, hormone,) molecule. The fluorophore-tagged antibody or fluorophore-tagged tumor avid moiety is responsive to the excitation wavelength administered to the subject. In another embodiment, the therapeutic drug is any compound or chemical, commonly accepted for therapeutic use in a patient with cancer

In another embodiment, the present invention provides methods for utilizing a diagnostic procedure during surgery in a subject in need thereof. In this embodiment of the invention diagnostic methods, an in vivo body part (e.g., tissue or organ) of the subject containing diseased tissue is illuminated with light having at least one excitation wavelength in the range from about 401 nm to about 510 nm. The targeting construct is pre-administered to the subject and is specifically bound to and/or been taken up by the diseased tissue or organ in the body part. The targeting construct fluoresces in response to the at least one excitation wavelength and is directly viewed to determine the location and/or surface area of the diseased tissue in the subject. Because the fluorescence is directly viewed and is specifically bound to the diseased tissue, all or at least a portion of the diseased tissue can be removed. After excision of all visible diseased or malignant tissue (aided by induced tumor fluorescence), clusters of microscopic cancer cells will be eliminated by the therapeutic drug bound to the targeting construct. The targeting construct comprises a fluorophore-tagged antibody or fluorophore-tagged tumor avid moiety and a therapeutic drug molecule.

In yet another embodiment, the present invention provides methods for in vivo diagnosis of tumor tissue in a subject in need thereof. In this embodiment, the invention method includes contacting samples of tumor cells obtained from the subject in vitro with a plurality of detectably labeled compounds, each of which binds to or is selectively taken up by a distinct tumor type to determine which of the compounds is bound to or taken up by the sample tumor cells. A biologically compatible fluorescing targeting construct is fabricated to contain a compound determined by this process that is tagged with a therapeutic drug molecule to bind to and/or be taken up by the sample tumor cells and which fluoresces in response to light having at least one excitation wavelength in the range from about 401 nm to about 510 nm. The location and/or surface area of the tumor tissue in the in vivo body part is diagnosed by administering a diagnostically effective amount of the targeting construct to the subject, allowing the targeting construct to bind to or be taken up by in vivo tumor cells, and directly viewing fluorescence emanating from the targeting construct bound to or taken up in the tumor tissue in response to illumination of the tumor tissue with a light that provides the required excitation wavelength.

In another embodiment, the present invention provides a means of eliminating microscopically small clusters of malignant cells that are not visible to the naked eye or with standard magnification devices by means of the delivery of therapeutic drug molecule attached to the fluorescent targeting construct. The therapeutic drug allows for destruction of the malignant cells.

### BRIEF DESCRIPTION OF THE DRAWINGS

FIG. 1 is an illustration depicting a methodology in an embodiment of the invention.

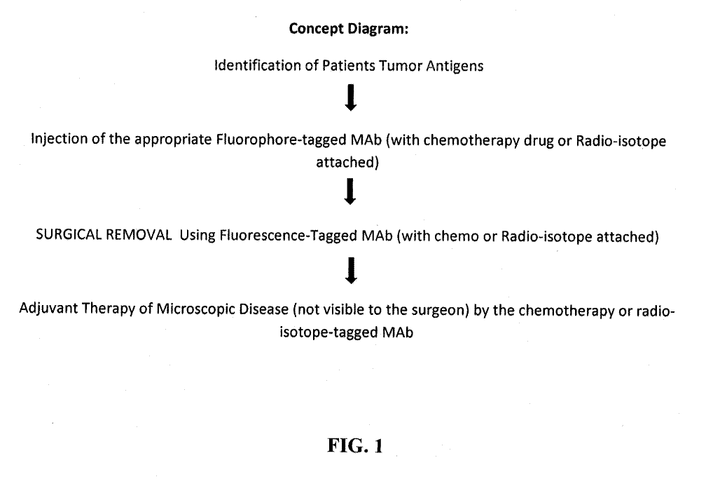

FIG. 2 is a diagram of an imaging device in an embodiment of the invention.

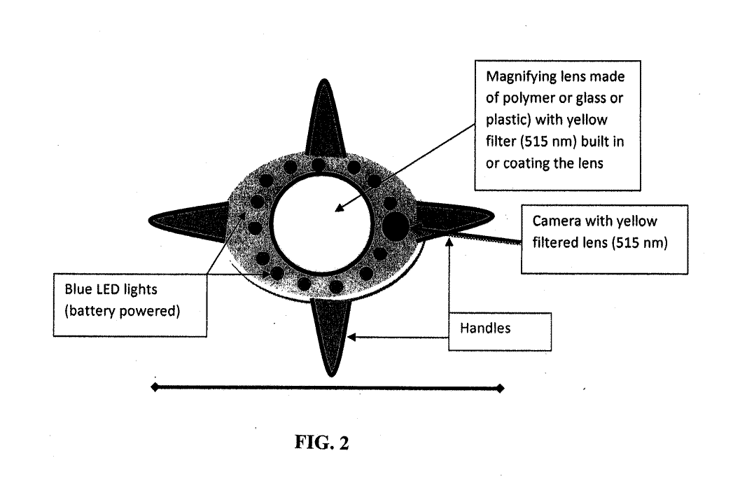

FIG. 3 is a diagram of an imaging device in an embodiment of the invention.

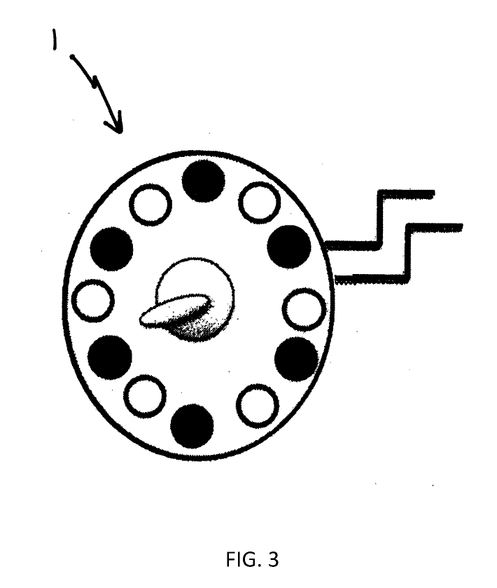

### DETAILED DESCRIPTION OF THE INVENTION

The present invention provides methods for in vivo identification, diagnosis, and therapy of diseased tissue in a subject in need thereof. The invention method includes illuminating an in vivo body part of the subject containing diseased tissue with light having at least one excitation wavelength in the range from about 401 nm to about 510 nm. Fluorescence emanating from a fluorescent targeting construct administered to the subject and which has specifically bound to and/or been taken up by the diseased tissue in the body part, in response to the at least one excitation wavelength is directly viewed to determine the location and/or surface area of the diseased tissue in the subject For other references see U.S. Pat. Nos. 4,444,744, 4,932,412, 5,697,902 and 7,011,812, the entire contents of which are incorporated herein by reference, for additional information regarding use of a radioisotope for therapy when attached to an antibody.

Light having a wavelength range from 401 nm to 510 nm lies within the visible range of the spectrum, in contrast to UV light, which lies within the non-visible range from about 4 nm to about 400 nm. Therefore, the excitation light used in practice of the invention diagnostic methods will contain at least one wavelength of light that illuminates surrounding tissue as well as excites fluorescence from the fluorescent targeting construct used in practice of the invention methods. The excitation light may be monochromatic or polychromatic. To compensate for the tendency of such background effect to obscure the desired visualization, a filter is used to screen out wavelengths below about 515 nm in the excitation light, thereby eliminating wavelengths that would be reflected from healthy tissue so as to cause loss of resolution of the fluorescent image. Alternatively, it is possible to view the diagnostic site through a filter that substantially screens out wavelengths other than the peak emission wavelength of the fluorophore used. For example, if the fluorescent targeting construct emits fluorescence at a known peak emission wavelength of 515-520 nm, the filter can be selected to substantially eliminate wavelengths of light below about 515 nm. Use of a filter in the practice of the invention diagnostic methods is expressly intended to be encompassed by the term “directly viewing” as applied to the invention diagnostic methods.

Use of one or more filters to screen out wavelengths of light in a selected wavelength band or screen out all wavelengths except those in a narrow band is well known in the art and will encompass the use of such simple devices as filtering eyeglasses worn by the diagnostician or physician, and/or filtered viewing lenses for endoscopic devices that are used during the diagnostic procedure.

Operating rooms can be equipped with an overhead light that emits wavelengths of light in the optical spectrum useful in practice of invention diagnostic methods, such as a Blue LED. Such a light can be utilized in the practice of the invention diagnostic methods merely by turning out the other lights in the operating room (to eliminate extraneous light that would be visibly reflected from tissue in the body part under investigation) and shining the excitation light into the body cavity or surgically created opening so that the fluorescent image received directly by the eye of the observer (e.g., the surgeon) is predominantly the fluorescent image emanating from the fluorophore(s) in the field of vision. Light emanating from a source in the 401-510 nm range could be filtered to aid in accomplishing the goal of direct visualization by the observer so that light reflecting from the body part, other than that from the fluorescing moiet(ies), is minimized or eliminated.

Light in the 401 nm to 510 nm wavelength range is readily absorbed in tissue. Accordingly, in the invention diagnostic methods, the diseased tissue (and bound or taken-up targeting construct) is “exposed” to the excitation light (e.g., by surgically created opening or endoscopic delivery of the light to an interior location. The invention method is particularly suited to in vivo detection of diseased tissue located at an interior site in the subject, such as within a natural body cavity or a surgically created opening, where the diseased tissue is “in plain view” (i.e., exposed to the human eye) to facilitate a procedure of biopsy or surgical excision, but would be equally applicable to visualizing malignant tissue of the skin or appendages. As the precise location and/or surface area of the tumor tissue are readily determined by the invention diagnostic procedure, the invention method is a valuable guide to the surgeon, who needs to “see” in real time the exact outlines, size, etc., of the diseased tissue or mass to be resected as the surgery proceeds. Once the diseased tissue is removed, any residual microscopic clusters of cells, with the therapeutic drug tumor-specific construct attached, would be destroyed by the therapeutic drug molecule contained within the fluorescent targeting construct.

If the putative diseased site is a natural body cavity or surgically produced interior site, an endoscopic device can be used to deliver the excitation light to the site, to receive fluorescence emanating from the site within a body cavity, and to aid in formation of a direct image of the fluorescence from the diseased tissue. For example, a lens in the endoscopic device can be used to focus on the detected fluorescence as an aid in visualizing the diseased tissue. As used herein, such endoscope-delivered fluorescence is said to be “directly viewed” by the practitioner and the tissue or organ to which the targeting construct binds or in which it is taken up must be “in plain view” to the endoscope since the light used in the invention diagnostic procedure will not contain wavelengths of light that penetrate tissue, such as wavelengths in the near infrared range. Alternatively, as described above, the excitation light may be directed by any convenient means, such as a hand-held LED or fixed light source, into a body cavity or surgical opening containing a targeting construct administered as described herein and the fluorescent image so produced can be directly visualized by the eye of the observer without aid from an endoscope. With or without aid from any type of endoscopic device, the fluorescence produced by the invention method is such that it can be viewed without aid of an image processing device, such as a CCD camera, TV monitor, photon collecting device, and the like.

In one embodiment of the invention diagnostic methods, diseased or abnormal tissues or organs are contemporaneously viewed through a surgical opening to facilitate a procedure of biopsy or surgical excision. As the location and/or surface area of the diseased tissue or organ are readily determined by the invention diagnostic procedure, the invention method is a valuable guide to the surgeon, who needs to know the exact outlines, size, etc., of the mass, for example, for resection as the surgery proceeds.

Accordingly, in this embodiment, the present invention provides methods for utilizing a diagnostic procedure during surgery in a subject in need thereof by irradiating an in vivo body part of the subject containing diseased tissue with light having at least one excitation wavelength in the range from about 401 nm to about 510 nm, directly viewing fluorescence emanating from a targeting construct administered to the subject that has specifically bound to and/or been taken up by the diseased tissue in the body part, wherein the targeting construct fluoresces in response to the at least one excitation wavelength, determining the location and/or surface area of the diseased tissue in the subject, and removing at least a portion of the tumor tissue.

In yet another embodiment, the present invention provides methods for in vivo diagnosis of tumor tissue in a subject in need thereof. In this embodiment, the invention method comprises contacting samples of tumor cells obtained from the subject in vitro with a plurality of detectably labeled compounds, each of which binds to or is selectively taken up by a distinct tumor type, determining which of the compounds is bound to or taken up by the sample tumor cells, administering a diagnostically effective amount of at least one biologically compatible fluorescing targeting construct containing a compound determined to bind to and/or be taken up by the sample tumor cells that is tagged with a therapeutic drug molecule and a fluorophore responsive to at least one wavelength of light in the range from about 401 nm to about 510 nm, and diagnosing the location and/or surface area of the tumor tissue in the in vivo body part by directly viewing fluorescence emanating from the targeting construct bound or taken up in the tumor tissue upon irradiation thereof with light providing the at least one excitation wavelength for the fluorescent targeting construct.

In one embodiment of the invention method, a single type of fluorescent moiety is relied upon for generating fluorescence emanating from the irradiated body part (i.e., from the fluorescent targeting construct that binds to or is taken up by diseased tissue). Since certain types of healthy tissue fluoresce naturally, in such a case it is important to select a fluorescent moiety for the targeting construct that has a predominant excitation wavelength that does not contain sufficient wavelengths in the visible range of light to make visible the surrounding healthy tissue and thus inhibit resolution of the diseased tissue. Therefore, the light source used in practice of this embodiment of the invention emits light in the range from about 401 nm to about 510 nm. Thus, the methods of the invention involve contact of diseased tissue with a fluorescent targeting construct.

Exemplary fluorescent targeting constructs include anti-tumor antigen antibodies (e.g., FAB fragment, bispecific antibodies, diabodies, or antibody fragments) or tumor avid Compounds (e.g. deoxyglucose, methionine, somatostatin, hormones, hormone receptor ligands) and a biologically compatible fluorescing moiety. As used herein, the terms “fluorophore-tagged antibody” and “fluorophore-tagged tumor avid compound” respectively refer to such fluorescent targeting constructs that are responsive to specific excitation wavelengths administered to a subject in need of the methods of the invention.

In another embodiment, the fluorescent targeting construct is additionally tagged with a therapeutic drug molecule (e.g. chemotherapy drug, hormone, etc). The advantage of including a therapeutic drug molecule is that when attached to the fluorophore-tagged antibody or the fluorophore-tagged tumor avid compound, they provide the dual roles of (i) allowing for intra-operative visual imaging (direct viewing using tumor fluorescence) as a guide for the operating surgeon in accurately determining the location of the tumor or diseased tissue, and (ii) post-surgery “cleanup” (adjuvant therapy) of any microscopic clusters of tissue or cells that are too small to be seen by the surgeon, but could be a source of local and distant recurrences of the disease/cancer. Exemplary therapeutic drugs include but are not limited to, the classes of drugs shown in Table 1A below:

TABLE 1A

Alkylating agents
Anthracyclines
Cytoskeletal disruptors
Epothilones
Inhibitors of topoisomerase II
Nucleotide analogs and precursor analogs
Peptide antibiotics
Platinum-based agents
Vinca alkaloids and derivatives and tubulin inhibitors
Retinoids
Tyrosine kinase inhibitors
BRAF inhibitors
MEK inhibitors
mTOR inhibitors
Growth factor inhibitors
Hormones

In another embodiment, the fluorescent targeting construct is additionally tagged with a therapeutic isotope molecule that is both an electron emitter and a positron (+) “beta” emitter. See U.S. Pat. No. 6,667,024, the entire content of which is incorporated herein by reference, for additional information regarding use of alpha or beta emitters for therapeutic use. The advantage of including a therapeutic isotope molecule is that when attached to the fluorophore-tagged antibody or the fluorophore-tagged tumor avid compound, they provide the dual roles of (i) allowing for pre-surgery external imaging with a positron emission tomography (PET) scanner of the subject to provide additional information and/or a guide for the operating surgeon in accurately determining the location of the tumor or diseased tissue, and (ii) post-surgery “cleanup” of any microscopic clusters of tissue or cells that are too small to be seen by the surgeon, but could be a source of local and distant recurrences of the disease/cancer. The dual emitter therapeutic isotopes provide the added benefit of providing short half-lives, thereby providing minimal risk of radiation exposure to the surgeon during the procedure. Exemplary therapeutic isotopes include, but are not limited to, those shown in Table 1B below:

TABLE 1B

| Therapeutic Isotope | Half Life | |
|---|---|---|
| Astatine-211 | 7 | h |
| Bismuth-213 | 46 | min |
| Carbon-11 | 20.38 | min |
| Chromium-51 | 28 | d |
| Cobalt-57 | 272 | d |
| Cobalt-60 | 10.5 | months |
| Copper-64 | 13 | h |
| Dysprosium-165 | 2 | h |
| Erbium-169 | 9.4 | d |
| Fluorine-18 | 1.8 | h |
| Gallium-67 | 78 | h |
| Holmium-166 | 26 | h |
| Indium-111 | 2.8 | d |
| Iodine-123 | 13 | h |
| Iodine-125 | 60 | d |
| Iodine-131 | 8 | d |
| Iridium-192 | 74 | d |
| Iron-59 | 46 | d |
| Krypton-81m | 13 | sec |
| Lutetium-177 | 6.7 | d |
| Molybdenum-99 | 66 | h |
| Nitrogen-13 | 10 | min |
| Oxygen-15 | 2 | min |
| Palladium-103 | 17 | d |
| Phosphorus-32 | 14 | d |
| Potassium-42 | 12 | h |
| Rhenium-186 | 3.8 | d |
| Rhenium-188 | 17 | h |
| Rubidium-81 | 4.6 | h |
| Rubidium-82 | 65 | h |
| Samarium-153 | 47 | h |
| Selenium-75 | 120 | d |
| Sodium-24 | 15 | h |
| Strontium-89 | 50 | d |
| Strontium-92 | 25 | d |
| Thallium-201 | 73 | h |
| Technetium-99m | 6 | h |
| Terbium-149 | 4.3 | min |
| Xenon-133 | 5 | d |
| Ytterbium-169 | 32 | d |
| Ytterbium-177 | 1.9 | h |
| Yttrium-90 | 64 | h |

The fluorescing moiety of the targeting construct can be any chemical or protein moiety that is biologically compatible (e.g., suitable for in vivo administration) and which fluoresces in response to excitation light as described herein. Since the targeting ligand is administered to living tissue, biological compatibility includes the lack of substantial toxic effect to the individual in general if administered systemically, or to the target tissue, if administered locally, at the dosage administered. Non limiting examples of fluorophores that can be used in the practice of the invention include fluorescein, fluorescein derivatives, tetracycline, quinine, mithramycin, Oregon green, and cascade blue, and the like, and combinations of two or more thereof.

Additional non-limiting examples of fluorescent compounds that fluoresce in response to an excitation wavelength in the range from 401 nm to about 510 nm are found in Table 2 below:

TABLE 2

| | EXCITATION | EMISSION |
|---|---|---|
| COMPOUND | RANGE (nm) | RANGE (nm) |
| Acridine Red | 455-600 | 560-680 |
| Acridine Yellow | 470 | 550 |
| Acriflavin | 436 | 520 |
| AFA (Acriflavin Feulgen SITSA) | 355-425 | 460 |
| Alexa Fluor 488 | 470-490 | 520 |
| ACMA | 430 | 474 |
| Astrazon Orange R | 470 | 540 |
| Astrazon Yellow 7 GLL | 450 | 480 |
| Atabrine | 436 | 490 |
| Auramine | 460 | 550 |
| Aurophosphine | 450-490 | 515 |
| Aurophosphine G | 450 | 580 |
| Berberine Sulphate | 430 | 550 |
| BOBO-1, BO-PRO-1 | 462 | 481 |
| BOPRO1 | 462 | 481 |
| Brilliant Sulpho-flavin FF | 430 | 520 |
| Calcein | 494 | 517 |
| Calcofluor White | 440 | 500-520 |
| Cascade Blue | 400 | 425 |
| Catecholamine | 410 | 470 |
| Chinacrine | 450-490 | 515 |
| Coriphosphine O | 460 | 575 |
| DiA | 456 | 590 |
| Di-8-ANEPPS | 488 | 605 |
| DiO [DiOC₁₈(3)] | 484 | 501 |
| Diphenyl Brilliant Flavine 7GFF | 430 | 520 |
| Euchrysin | 430 | 540 |
| Fluorescein | 494 | 518 |
| Fluorescein Iso-thiocyanate (FITC) | 490 | 525 |
| Fluo 3 | 485 | 503 |
| FM1-43 | 479 | 598 |
| Fura Red | 472 (low[Ca²⁺]) | 657 (low[Ca²⁺]) |
| | 436 (high[Ca²⁺]) | 637 (high[Ca²⁺]) |
| Genacryl Brilliant Yellow 10GF | 430 | 485 |
| Genacryl Pink 3G | 470 | 583 |
| Genacryl Yellow SGF | 430 | 475 |
| Gloxalic Acid | 405 | 460 |
| 3-Hydroxypyrene-5,-8,10- | 403 | 513 |
| TriSulfonic Acid | | |
| 7-Hydroxy-4-methylcourmarin | 360 | 455 |
| 5-Hydroxy-Tryptamine (5-HT) | 380-415 | 520-530 |
| Lucifer Yellow CH | 425 | 528 |
| Lucifer Yellow VS | 430 | 535 |
| LysoSensor Green DND-153, | 442 | 505 |
| DND-189 | | |
| Maxilon Brilliant Flavin 10 GFF | 450 | 495 |
| Maxilon Brilliant Flavin 8 GFF | 460 | 495 |
| Mitotracker Green FM | 490 | 516 |
| Mithramycin | 450 | 570 |
| NBD | 465 | 535 |
| NBD Amine | 450 | 530 |
| Nitrobenzoxadidole | 460-470 | 510-650 |
| Nylosan Brilliant Flavin E8G | 460 | 510 |
| Oregon Green 488 fluorophore | 496 | 524 |
| Phosphine 3R | 465 | 565 |
| Quinacrine Mustard | 423 | 503 |
| Rhodamine 110 | 496 | 520 |
| Rhodamine 5 GLD | 470 | 565 |
| Rhodol Green fluorophore | 499 | 525 |
| Sevron Orange | 440 | 530 |
| Sevron Yellow L | 430 | 490 |
| SITS (Primuline) | 395-425 | 450 |
| Sulpho Rhodamine G Extra | 470 | 570 |
| SYTO Green fluorescent | 494 ± 6 | 515 ± 7 |
| nucleic acid stains | | |
| Thioflavin S | 430 | 550 |
| Thioflavin 5 | 430 | 550 |
| Thiozol Orange | 453 | 480 |
| Uranine B | 420 | 520 |
| YOYO-1, YOYO-PRO-1 | 491 | 509 |

Since the fluorescence properties of biologically compatible fluorophores are well known, or can be readily determined by those of skill in the art, the skilled practitioner can readily select a useful fluorophore or useful combination of fluorophores, and match the wavelength(s) of the excitation light to the fluorophore(s). The toxicity of fluorescein is minimal as it has been used safely in vivo in humans for many years, but the toxicity of additional useful fluorophores can be determined using animal studies as known in the art.

Preferably, the targeting construct (e.g., the ligand moiety of the invention targeting construct) is selected to bind to and/or be taken up specifically by the target tissue of interest, for example to an antigen or other surface feature contained on or within a cell that characterizes a disease or abnormal state in the target tissue. As in other diagnostic assays, it is desirable for the targeting construct to bind to or be taken up by the target tissue selectively or to an antigen associated with the disease or abnormal state; however, targeting constructs containing ligand moieties that also bind to or are taken up by healthy tissue or cell structures can be used in the practice of the invention method so long as the concentration of the antigen in the target tissue or the affinity of the targeting construct for the target tissue is sufficiently greater than for healthy tissue in the field of vision so that a fluorescent image representing the target tissue can be clearly visualized as distinct from any fluorescence coming from healthy tissue or structures in the field of vision. For example, colon cancer is often characterized by the presence of carcinoembryonic antigen (CEA), yet this antigen is also associated with certain tissues in healthy individuals. However, the concentration of CEA in cancerous colon tissue is typically greater than is found in healthy tissue, so an anti-CEA antibody could be used as a ligand moiety in the practice of the invention. In another example, deoxyglucose is taken up and utilized by healthy tissue to varying degrees, yet its metabolism in healthy tissues, except for certain known organs, such as the heart, is substantially lower than in tumor. The known pattern of deoxyglucose consumption in the body can therefore be used to aid in determination of those areas wherein unexpectedly high uptake of deoxyglucose signals the presence of tumor cells.

Thus, in one embodiment, the disease or abnormal state detected by the invention method can be any type characterized by the presence of a known target tissue for which a specific binding ligand is known. For example, various heart conditions are characterized by production of necrotic or ischemic tissue or production of artherosclerotic tissue for which specific binding ligands are known. As another illustrative example, breast cancer is characterized by the production of cancerous tissue identified by monoclonal antibodies to CA15-3, CA19-9, CEA, or HER2/neu. It is contemplated that the target tissue may be characterized by cells that produce either a surface antigen for which a binding ligand is known, or an intracellular marker (i.e. antigen), since many targeting constructs penetrate the cell membrane. Representative disease states that can be identified using the invention method include such various conditions as different types of tumors, bacterial, fungal and viral infections, and the like. As used herein “abnormal tissue” includes precancerous conditions, cancer, necrotic or ischemic tissue, and tissue associated with connective tissue diseases, and auto-immune disorders, and the like. Further, examples of the types of target tissue suitable for diagnosis or examination using the invention method include cancer of breast, lung, colon, prostate, pancreas, skin, stomach, small intestine, testicle, head and neck, thyroid, gall bladder, brain, endocrine tissue, and the like, as well as combinations of any two or more thereof.

Representative examples of antigens for some common malignancies and the body locations in which they are commonly found are shown in Table 3 below. Targeting ligands, such as antibodies, for these antigens are known in the art.

TABLE 3

| | TUMORS WHERE |
|---|---|
| ANTIGEN | COMMONLY FOUND |
| CEA (carcinoembryonic | colon, breast, lung, pancreas |
| antigen) | |
| PSA (prostate specific | prostate cancer |
| antigen) | |
| PSMA (prostate specific | prostate cancer |
| membrane antigen) | |
| CA-125 | ovarian cancer, breast, colon, lung |
| CA 15-3 | breast cancer, lung, colon, pancreas, |
| | medullary cancer of the thyroid, prostate |
| CA 19-9 | breast cancer |
| HER2/neu | breast cancer |
| α-feto protein | testicular cancer, hepatic cancer |
| β-HCG | testicular cancer, choriocarcinoma (human |
| | chorionic gonadotropin) |
| MUC-1 | breast cancer, colon, lung, |
| MUC-2 | colorectal cancer, colon, lung |
| TAG 72 | breast cancer, colon cancer, and pancreatic |
| | cancer |
| Estrogen receptor | breast cancer, uterine cancer |
| Progesterone receptor | breast cancer, uterine cancer |
| AR (androgen receptor) | prostate cancer |
| EGFr (epidermal growth | bladder cancer |
| factor receptor) | |
| IGFr (insulin like growth | Sarcoma |
| factor) | |

In one embodiment of the invention method, the ligand moiety of the targeting construct is a protein or polypeptide, such as an antibody, or biologically active fragment thereof, preferably a monoclonal antibody. The supplemental fluorescing targeting construct(s) used in practice of the invention method may also be or comprise polyclonal or monoclonal antibodies tagged with a fluorophore. The term “antibody” as used in this invention includes intact molecules as well as functional fragments thereof, such as Fab, F(ab′)₂, and Fv that are capable of binding the epitopic determinant. These functional antibody fragments retain some ability to selectively bind with their respective antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)_{2,} the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow & Lane, Antibodies: A Laboratory Manual, Cold Spring Harbor Laboratory, New York, 1988, incorporated herein by reference). As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments of the present invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)_{2.} This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated in their entireties by reference. See also Nisonhoff et al., Arch. Biochem. Biophys. 89:230, 1960; Porter, Biochem. J. 73:119, 1959; Edelman et al., Methods in Enzymology, Vol. 1, page 422 Academic Press, 1967; and Coligan et al. at sections 2.8.1-2.8.10 and 2.10.1-2.10.4. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659, 1972. Alternatively, the variable chains can be linked by an intermolecular disulfide bond or crosslinked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., Methods: a Companion to Methods in Enzymology, 2: 97, 1991; Bird et al., Science 242:423-426, 1988; Pack et al., Bio/Technology 11:1271-77, 1993; Sandhu, supra, and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: a Companion to Methods in Enzymology, 2: 106, 1991.

Antibodies which bind to a tumor cell can be prepared using an intact polypeptide or biologically functional fragment containing small peptides of interest as the immunizing antigen. The polypeptide or a peptide used to immunize an animal (derived, for example, from translated cDNA or chemical synthesis) can be conjugated to a carrier protein, if desired. Commonly used carriers that are chemically coupled to the peptide include keyhole limpet hemocyanin (KLH), thyroglobulin, bovine serum albumin (BSA), and tetanus toxoid, and the like. The coupled peptide is then used to immunize the animal (e.g., a mouse, a rat, or a rabbit).

The preparation of such monoclonal antibodies is conventional. See, for example, Kohler & Milstein, Nature 256:495, 1975; Coligan et al., sections 2.5.1-2.6.7; and Harlow et al., in: Antibodies: a Laboratory Manual, page 726 (Cold Spring Harbor Pub., 1988), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104 (Humana Press, 1992).

Antibodies of the present invention may also be derived from subhuman primate antibodies. General techniques for raising therapeutically useful antibodies in baboons can be found, for example, in Goldenberg et al., International Patent Publication WO 91/11465 (1991) and Losman et al., 1990, Int. J. Cancer 46:310, which are hereby incorporated by reference. Alternatively, a therapeutically useful antibody may be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833,1989, which is hereby incorporated in its entirety by reference. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Nat'l Acad. Sci. USA 89:4285, 1992; Sandhu, Crit. Rev. Biotech. 12:437, 1992; and Singer et al., J. Immunol. 150:2844, 1993, which are hereby incorporated by reference.

A variety of methods are available for the production of monoclonal antibodies (see Of mice and men: hybridoma and recombinant antibodies. Immunol Today, Little M, Kipriyanov S M, Le Gall F, Moldenhauer G., August; 21 (8): 364-70, 2000), and include the production of fully human monoclonal antibodies from rabbit hybridomas, for example in Pytela, et al., U.S. Pat. No. 7,429,487, and U.S. Pat. No. 8,062,867.

It is also possible to use anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

In a presently preferred embodiment of the invention method, the ligand moiety in the fluorescent targeting construct used in practice of the invention can be selected from among the many biologically compatible tumor-avid moieties that bind with specificity to receptors and/or are preferentially taken up by tumor cells, and can be used as the ligand moiety in the invention targeting constructs. Tumor-avid moieties that are preferentially “taken up” by tumor cells may enter the cells through surface or nuclear receptors (e.g., hormone receptors), pores, hydrophilic “windows” in the cell lipid bilayer, and the like.

Illustrative of this class of tumor-avid moieties are somatostatin, somatostatin receptor-binding peptides, deoxyglucose, methionine, histidine, folic acid, and the like. Particularly useful somatostatin receptor-binding peptides are a long-acting, octapeptide analog of somatostatin, known as octreotide (D-phenylalanyl-L-cysteinyl-L-phenylalanyl-D-tryptophyl-L-lysyl-Lthreonyl-N-[2-hydroxy-1-(hydroxymethyl)propyl]-L-cysteinamide cyclic (2.fwdarw.7)-disulfide), lanreotide, an oral formulation of octreotide, P829, P587, and the like. Somatostatin binding peptides are disclosed in U.S. Pat. No. 5,871,711, and methods for linking such peptides covalently to a radioisotope through their carboxyl terminal amino acid under reducing conditions are disclosed in U.S. Pat. No. 5,843,401, which are both incorporated herein by reference in their entireties. One of skill in the art can readily adapt such teachings for the preparation of fluorescence-sensitive somatostatin receptor-binding peptides by substituting the fluorescing moieties of this invention in the place of a radioisotope.

Somatostatin and somatostatin receptor-binding peptides are particularly effective for use as the tumor-avid moiety in the targeting construct in the invention diagnostic procedures when the disease state is a neuroendocrine or endocrine tumor. Examples of neuroendocrine tumors that can be diagnosed using the invention method include adenomas (GH-producing and TSH-producing), islet cell tumors, carcinoids, undifferentiated neuroendocrine carcinomas, small cell and non small cell lung cancer, neuroendocrine and/or intermediate cell carcinomas, neuroendocrine tumors of ovary, cervix, endometrium, breast, kidney, larynx, paranasal sinuses, and salivary glands, meningiomas, well differentiated glia-derived tumors, pheochromocytomas, parathyroid adenomas, neuroblastomas, ganglioneuro(blasto)mas, paragangliomas, papillary, follicular and medullary carcinomas in thyroid cells, Merkel cell carcinomas, and melanomas, as well as granulomas and lymphomas. These tumor cells are known to have somatostatin receptors and can be targeted using somatostatin or somatostatin receptor binding peptides as the tumor-avid moiety in the invention fluorescent targeting construct.

Vasointestinal peptide (VIP), which is used in VIP receptor scintigraphy (I. Virgolini, Eur J. Clin. Invest. 27(10):793-800, 1997), is also useful in the invention method for diagnosis of small primary adenocarcinomas, liver metastases and certain endocrine tumors of the gastrointestinal tract.

Another molecule illustrative of the tumor-avid moieties that are preferentially taken up by tumors is deoxyglucose, which is known to be preferentially taken up in a variety of different types of tumors. Illustrative of the types of tumors that can be detected using deoxyglucose as the tumor-avid ligand moiety in the fluorescent targeting construct as disclosed herein include Preferred tumor targets for deoxyglucose include melanoma, colorectal and pancreatic tumors, lymphoma (both HD and NHL), head and neck tumors, myeloma, cancers of ovary, cancer, breast, and brain (high grade and pituitary adenomas), sarcomas (grade dependent), hepatoma, testicular cancer, thyroid (grade dependent) small cell lung cancer, bladder and uterine cancer, and the like.

Yet other tumor-avid compounds that can be used as the targeting ligand in an invention fluorescing targeting construct are 1-amino-cyclobutane-1-carboxylic acid and Lmethionine. L-methionine is an essential amino acid that is necessary for protein synthesis. It is known that malignant cells have altered methionine metabolism and require an external source of methionine.

Additional examples of biologically compatible tumor-avid compounds that bind with specificity to tumor receptors and/or are preferentially taken up by tumor cells include mammalian hormones, particularly sex hormones, neurotransmitters, and compounds expressed by tumor cells to communicate with each other that are preferentially taken up by tumor cells, such as novel secreted protein constructs arising from chromosomal aberrations, such as transfers or inversions within the clone.

The term “hormone” is used herein to refer to compounds that are expressed within a mammal for action at a remote location and includes such compounds as sex hormones, cell growth hormones, cytokines, endocrine hormones, erythropoietin, and the like. As is known in the art, a number of tumor types express receptors for hormones, for example, estrogen, progesterone, androgens, such as testosterone, and the like. Such hormones are preferentially taken up by tumor cells, for example, via specific receptors. It is also known in the art that the particular type of receptors expressed by a tumor cell may change over time with the same cell or cell mass, for example, expressing estrogen receptors at one point in time and with the estrogen receptors being substantially replaced with androgen receptors at another point in time.

Therefore, in another embodiment according to the present invention, the invention diagnostic method comprises prescreening of target tumor cells to determine which receptors are currently being expressed by the target cells. In this embodiment, the invention diagnostic method comprises contacting sample(s) of tumor cells obtained from a subject in vitro with a plurality of detectably labeled tumor-avid compounds, and determining which of the tumor-avid compounds bind to or are taken up by the sample cells. The invention diagnostic method further comprises administering to the subject a diagnostically effective amount of one or more biologically compatible fluorescing targeting constructs, each comprising as ligand moiety at least one of the tumor-avid compounds determined to bind to and/or be taken up by the tumor cells so as to allow the fluorescing targeting construct to bind to and/or be taken up selectively in vivo by tumor tissue, irradiating an in vivo body part of the subject suspected of containing the tumor tissue with light having at least one wavelength in the excitation spectrum of the targeting construct under conditions that substantially eliminate extraneous light to the in vivo body part, and directly viewing fluorescence emanating from the fluorescing targeting construct bound to or taken up by the tumor tissue so as to determine the location and/or surface area of the tumor tissue in the in vivo body part. Of course, if the tests determine that the tumor cells are concurrently taking up more than one tumor-avid compound in substantial proportion (e.g., both estrogen and progesterone), the more than one tumor avid compound so determined can be used as the tumor-avid ligand moieties in the targeting constructs in the invention diagnostic method.

Methods for obtaining test tumor cells for prescreening to determine the type(s) of tumor-avid compounds that are currently being taken up (e.g., by specific receptors expressed by the tumor cells) are well known in the art. For example, such techniques as fine needle aspirates, brush biopsies, core needle biopsies, pleural effusion, ascetic fluid urine and sputum cytology, bone marrow biopsy and aspirates, scrapings, excisional biopsies, and the like, can in many instances be utilized to obtain test tumor cells relatively non-invasively.

In vitro tests useful for determining the tumor-avid compounds that are being taken up by test tumor cells are numerous and also well known in the art. Such in vitro tests generally involve either sequentially or simultaneously contacting the test cells with a plurality of different tumor-avid compounds. For example, the test cells can be contacted with a panel or library of detectably labeled hormones and/or other known tumor-avid compounds to determine which of the detectably labeled compounds bind to and/or are taken up by the test cells.

In the practice of the present invention, the fluorescent moiety sensitive to an excitation wavelength in the 401 nm to 510 nm range can be linked to the tumor-avid compound used as the ligand moiety in the targeting construct by any method presently known in the art for attaching two moieties, so long as the attachment of the linker moiety to the ligand moiety does not substantially impede binding of the targeting construct to the target tissue and/or uptake by the tumor cells, for example, to a receptor on a cell. Those of skill in the art will know how to select a ligand/linker pair that meets this requirement. For example, with regard to octreotide, it has been shown that coupling of a linker to Tyr3 or Phe1 of octreotide does not prevent the internalization of octreotide after binding to the somatostatin receptor (L. J. Hofland et al., Proc. Assoc. Am. Physicians 111:63-9, 1999). It is also known that 1-amino-cyclobutane-1-carboxylic acid can be tagged at the 3 carbon of the ring.

The length of the optional linker moiety is chosen to optimize the kinetics and specificity of ligand binding, including any conformational changes induced by binding of the ligand moiety to a target, such as an antigen or receptor. The linker moiety should be long enough and flexible enough to allow the ligand moiety and the target to freely interact and not so short as to cause steric hindrance between the proteinaceous ligand moiety and the target.

In one embodiment, the linker moiety is a heterobifunctional cleavable cross-linker, such as N-succinimidyl (4-iodoacetyl)-aminobenzoate; sulfosuccinimidyl(4-iodoacetyl)-aminobenzoate; 4-succinimidyl-oxycarbonyl-.alpha.-(2-pyridyldithio) toluene; sulfosuccinimidyl-6-[.alpha.-methyl.alpha.-(pyridyldithiol)-toluamido]hexanoate; Nsuccinimidyl-3-(-2-pyridyldithio)-proprionate; succinimidyl-6-[3(-(-2-pyridyldithio)-proprionamido]hexanoate; sulfosuccinimidyl-6-[3(-(-2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking compounds are disclosed in U.S. Pat. Nos. 5,349,066, 5,618,528, 4,569,789, 4,952,394, and 5,137,877, each of which is incorporated herein by reference in its entirety.

These chemical linkers can be attached to purified ligands using numerous protocols known in the art, such as those described in Pierce Chemicals “Solutions, Cross-linking of Proteins: Basic Concepts and Strategies,” Seminar #12, Rockford, Ill.

In another embodiment presently preferred, the linker moiety is a peptide having from about 2 to about 60 amino acid residues, for example from about 5 to about 40, or from about 10 to about 30 amino acid residues. This alternative is particularly advantageous when the ligand moiety is proteinaceous. For example, the linker moiety can be a flexible spacer amino acid sequence, such as those known in single-chain antibody research. Examples of such known linker moieties include GGGGS (SEQ ID NO:1), (GGGGS)ₙ (SEQ ID NO:2), GKSSGSGSESKS (SEQ ID NO:3), GSTSGSGKSSEGKG (SEQ ID NO:4), GSTSGSGKSSEGSGSTKG (SEQ ID NO:5), GSTSGSGKSSEGKG (SEQ ID NO:6), GSTSGSGKPGSGEGSTKG (SEQ ID NO:7), EGKSSGSGSESKEF (SEQ ID NO:8), SRSSG (SEQ ID NO:9), SGSSC (SEQ ID NO:10), and the like. A Diphtheria toxin trypsin sensitive linker having the sequence AMGRSGGGCAGNRVGSSLSCGGLNLQAM (SEQ ID NO:11) is also useful. Alternatively, the peptide linker moiety can be VM or AM, or have the structure described by the formula: AM(G₂ to ₄S)ₙ XAM wherein X is selected from any amino acid and n is an integer from 1 to 11 (SEQ ID NO:12). Additional linking moieties are described, for example, in Huston et al., PNAS 85:5879-5883, 1988; Whitlow, M., et al., Protein Engineering 6:989-995, 1993; Newton et al., Biochemistry 35:545-553, 1996; A. J. Cumber et al., Bioconj. Chem. 3:397-401, 1992; Ladurner et al., J. Mol. Biol. 273:330-337, 1997; and U.S. Pat. No. 4,894,443, the latter of which is incorporated herein by reference in its entirety.

The targeting constructs and supplemental targeting constructs used in practice of the invention method can be administered by any route known to those of skill in the art, such as intravenously, intraarticularly, intracisternally, intraocularly, intraventricularly, intrathecally, intramuscularly, intraperitoneally, intradermally, intracavitarily, and the like, as well as by any combination of any two or more thereof.

The most suitable route for administration will be intravenously, but may vary depending upon the disease state to be treated, or the location of the suspected condition or tumor to be diagnosed.

The targeting construct is administered in a “diagnostically effective amount.” As used herein, a “diagnostically effective amount” refers to the quantity of a targeting construct necessary to aid in direct visualization of any target tissue located in the body part under investigation in a subject. As used herein, the term “subject” refers to any mammal, such as a domesticated pet, farm animal, or zoo animal, but preferably is a human. Amounts effective for diagnostic use will, of course, depend on the size and location of the body part to be investigated, the affinity of the targeting construct for the target tissue, the type of target tissue, as well as the route of administration.

Since individual subjects may present a wide variation in severity of symptoms and each targeting construct has its unique diagnostic characteristics, including, affinity of the targeting construct for the target, rate of clearance of the targeting construct by bodily processes, the properties of the fluorophore contained therein, and the like, the skilled practitioner will weigh the factors and vary the dosages accordingly.

The invention composition can also be formulated as a sterile injectable suspension according to known methods using suitable dispersing or wetting agents and suspending agents. The sterile injectable preparation may also be a sterile injectable solution or suspension in a nontoxic parenterally-acceptable diluent or solvent, for example, as a solution in 1-4, butanediol. Sterile, fixed oils are conventionally employed as a solvent or suspending medium. For this purpose any bland fixed oil may be employed, including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate, or the like. Buffers, preservatives, antioxidants, and the like, can be incorporated as required, or, alternatively, can comprise the formulation.

The invention fluorescing targeting constructs can be produced by well known techniques. For example, well known techniques of protein synthesis can be used to obtain proteinaceous components of the targeting construct if the amino acid sequence of the component is known, or the sequence can first be determined by well known methods, if necessary. Some of the ligand genes are now commercially available. An advantage of obtaining commercially available genes is that they have generally been optimized for expression in E. coli. A polynucleotide encoding a protein, peptide or polynucleotide of interest, can be produced using DNA synthesis technology. Methods for obtaining the DNA encoding an unavailable gene and expressing a gene product therefrom are well known and will not be described here in detail.

A fluorescent targeting construct comprising a proteinaceous ligand moiety, a proteinaceous linker moiety, and a proteinaceous fluorophore can also be produced as a fusion protein using well known techniques wherein a host cell is transfected with an expression vector containing expression control sequences operably linked to a nucleic acid sequence coding for the expression of the fusion protein (Molecular Cloning A Laboratory Manual, Sambrook et al., eds., 2nd Ed., Cold Spring Harbor Laboratory, N.Y., 1989).

As used herein, the terms “peptide” and “polypeptide” refer to a polymer in which the monomers are amino acid residues which are joined together through amide bonds, alternatively referred to as a polypeptide. When the amino acids are alpha-amino acids, either the L-optical isomer or the D-optical isomer can be used, the L-isomers being preferred. Additionally, unnatural amino acids such as beta-alanine, phenylglycine, and homoarginine are meant to be included. Commonly encountered amino acids that are not gene-encoded can also be used in the present invention, although preferred amino acids are those that are encodable. For a general review, see, for example, Spatola, A. F., in Chemistry and Biochemistry of Amino Acids, Peptides and Proteins, B. Weinstein, ed., Marcel Dekker, New York, p. 267, 1983.

### DEFINITIONS

As discussed in the body of the invention, the following terms apply

Monoclonal Antibody (including fully human, humanized, chimeric and also including whole antibodies, partial antibodies, Fab fragment, bispecific antibodies, diabodies, or antibody fragments, etc.)

Fluorophore (any non-toxic substance with excitation spectra in the visible light range (401-510 nm) and with emission spectra in the visible range (520-580 nm) with examples being fluorescein and fluorescein like derivatives, antibiotics (i.e. tetracycline), quinine, as well as quantum dots)

Radioisotopes: (any one of several radioisotopes to include but not limited to Cu-64, Rhenium-188, and Samarium-153

Therapeutic Drug (chemotherapy . . . to include all classes of commonly accepted chemotherapy i.e. anti-metabolites, antibiotics, DNA scission, anthracyclines, spindle cell inhibitors, proteasomes, mTOR inhibitors, tyrosine kinase inhibitors, hormones, HDAC inhibitors, epithilones, kinase inhibitors, etc.)

Diseased Tissue (to include cancer, endocrine adenomas, benign tumors with systemic effects)

Schematically it would be as follows:
Diagnosis of a potentially resectable and surgically curable cancer:
Identification of surface or internal antigens on or within the tumor or other diseased cells:
Injection of fluorophore-tagged (and chemotherapy-tagged or chemotherapy/radioisotope-tagged anti-tumor antigen MAb:
Surgical resection of all visibly fluorescent tumor tissue (1-5 days after injection of the MAb):
Adjuvant Therapy; destruction of microscopic (and not visible) residual cancer cells through the attached fluorophore-tagged+chemotherapy or chemotherapy/radioisotope-tagged MAb

Although the invention has been described with reference to the above example, it will be understood that modifications and variations are encompassed within the spirit and scope of the invention. Accordingly, the invention is limited only by the following claims.

1. A method, comprising:
(a) treating a subject having cancer with a construct comprising:
a fluorophore-tagged tumor-specific antibody or a fluorophore-tagged tumor avid moiety bound to a chemotherapeutic molecule, wherein the fluorophore-tagged antibody or fluorophore-tagged tumor avid moiety, such that the construct becomes specifically bound to or is taken up cancer tissue in the patient;
(b) illuminating an in vivo exposed body cavity, integument, or organ of the subject containing cancer tissue with light having at least one excitation wavelength in the range from 401 nm to 510 nm; and
(b) directly viewing the illuminated cavity, integument, or organ with or without the aid of an endoscope, to detect fluorescence emanating from the fluorophore bound to the cancer tissue in response to the illumination with the light;
(c) determining by direct visualization without the aid of a capture device the location and/or surface area of the diseased tissue in the subject from the fluorescence provided by the targeting construct; and thereafter
(d) leaving the chemotherapeutic molecule in contact with the cancer tissue to kill at least a portion of the cancer tissue.

2. The method of claim 1, wherein the fluorophore-tagged antibody is an anti-tumor antigen antibody, FAB fragment, bispecific antibody, or a functional antibody fragment thereof.

3. The method of claim 1, wherein the chemotherapeutic molecule is a chemotherapy compound selected from the group consisting of antimetabolites, antibiotics, anthracyclines, tubulin inhibitors, taxanes, mTOR inhibitors, tyrosine kinase inhibitors, histone deacetylase inhibitors, epithilones, multi-kinase inhibitors, and derivatives thereof.

4. The method of claim 1, wherein the excitation light has a peak intensity below 510 nm.

5. The method of claim 1, wherein the tumor-avid moiety is a hormone, a hormone receptor, hormone receptor binding-peptide, deoxyglucose, somatostatin, a somatostatin receptor-binding peptide, methionine, histidine, folic acid, cinacalcet, chromagranin, or a combination of any two or more thereof.

6. The method of claim 5, wherein the tumor-avid moiety is HER2 neu, an estrogen receptor, a progesterone receptor, an androgen receptor, a TSH receptor, a calcium sensing receptor, vascular endothelial growth factor receptor, insulin-like receptor, or platelet-derived growth factor receptor.

7. The method of claim 5, wherein the tumor-avid moiety is chromagranin, somatostatin, the somatostatin receptor-binding peptide, or cinacalcet.

8. The method of claim 1, wherein the cancer is thyroid cancer, parathyroid adenoma, adrenal cancer, islet cell cancer, pituitary adenoma, insulinoma, or pinealoma.

9. The method of claim 8, wherein the cancer is malignant melanoma, head and neck cancer, hepatic cancer, breast cancer, pancreas cancer, small cell and non-small cell lung cancer, ovarian cancer, endometrial cancer, or fallopian tube cancer, pituitary or adrenal cancer, brain cancer, esophageal cancer, gastric cancer, small bowel or colorectal cancer, anal cancer; basal cell cancer, squamous cell cancer of the skin, epithelial cancer, carcinoid tumor, prostate cancer, bladder cancer, renal cancer, vulvar cancer, testicular germ cell tumor, Ewing's sarcoma, soft tissue sarcoma, osteosarcoma, hemangiopericytoma, hemangiomatosis, or lymphangiomatosis.

10. The method of claim 5, wherein the tumor-avid moiety is a somatostatin receptor-binding peptide selected from the group consisting of octreotide, lanreotide, P587, and P829.

11. The method of claim 1, wherein the chemotherapeutic agent is deoxyglucose.

12. The method of claim 1, wherein the tumor-avid moiety is 1-amino-cyclobutane-1-carboxylic acid, methionine, a methionine derivative, or histidine.

13. The method of claim 1, further comprising surgically excising at least a part of the cancer tissue while directly viewing the illuminated cavity, integument, or organ.

14. The method of claim 1, further comprising determining the surface area of the cancer based on the intensity of the fluorescence.

15. The method of claim 1, wherein the light is monochromatic and the at least one excitation wavelength is matched to a predominant excitation wavelength of the fluorescent targeting construct.

16. The method of claim 15, wherein the at least one excitation wavelength is 400-510 nm and the fluorescent targeting construct comprises fluorescein, fluorescein derivatives, Alexa-Fluor 488, Hi-Lyte Fluor 488, quinine, or tetracycline.

17. The method of claim 1, wherein the exposed body cavity is a natural body cavity.

18. The method of claim 1, wherein the cancer is a muscle tumor, bone tumor, malignant mole, a thyroid mass, a brain tumor, a testicular cancer, or a prostate cancer.

19. The method of claim 1, wherein the body cavity is illuminated, and wherein the exposed body cavity is made surgically or the exposed body cavity is viewed through a rigid endoscope, a flexible endoscope, or through the use of robotic devices.

20. The method of claim 1, wherein a source of the light is located outside of the body of the subject.

21. The method of claim 1, wherein the viewing is for monitoring the course of the disease state.

22. The method of claim 1, wherein the viewing identifies the diseased tissue for surgical intervention.

23. The method of claim 1, further comprising surgically removing substantially all or at least a portion of the cancer tissue.

24. The method of claim 1, wherein the cancer tissue is associated with a condition selected from the group consisting of tumors, pre-cancerous conditions, or necrotic and ischemic conditions.

25. The method of claim 1, wherein the fluorescing targeting construct further comprises a linker moiety for attaching the antibody or the tumor avid moiety to the fluorescing moiety.

26. The method of claim 25, wherein the tumor-avid moiety is a hormone, deoxyglucose, somatostatin, a somatostatin receptor-binding peptide, methionine, a methionine derivative, or a combination of any two or more thereof.

27. The method of claim 1, wherein the targeting construct is administered by a method selected from the group consisting of intravenously, intramuscularly, intraarticularly, intracisternally, intraocularly, intraventricularly, intrathecally, intercavitarily, intraperitoneally, intradermally, and a combination of any two or more thereof.

28. The method of claim 1, wherein the targeting construct is administered by local injection.

29. The method of claim 1, wherein the targeting construct is administered systemically.

30. A method for utilizing a diagnostic procedure during surgery in a subject in need thereof comprising:
(a) illuminating an in vivo exposed body cavity or organ of the subject exposed by surgery and that contains diseased tissue with light having at least one excitation wavelength in the range from 401 nm to 510 nm;
(b) directly viewing with or without the aid of an endoscope or robotic device, a fluorescence emanating from a fluorescent targeting construct comprising:
an fluorophore-tagged antibody or a fluorophore-tagged tumor avid moiety, and
a chemotherapeutic molecule,
wherein the fluorophore-tagged antibody or fluorophore-tagged tumor avid moiety is responsive to the at least one excitation wavelength which is administered to the subject and which has specifically bound to and/or been taken up by the diseased tissue in the body cavity or organ, and wherein the targeting construct fluoresces in response to the at least one excitation wavelength;
(c) determining the location and/or surface area of the diseased tissue in the subject from the directly viewed fluorescence from the targeting construct; and
(d) removing all or at least a portion of the diseased tissue.

31. The method of claim 30, wherein the light has a peak intensity below 510 nm.

32. The method of claim 30, wherein the viewing of the fluorescence and the removing of the tumor tissue are performed substantially contemporaneously.

33. A method for in vivo diagnosis of tumor cells in a subject in need thereof comprising:
(a) contacting samples of tumor cells obtained from the subject in vitro with a plurality of detectably labeled compounds, each of which binds to one or more cell-surface tumor antigens or is selectively taken up by a distinct tumor type;
(b) determining which of the compounds is bound to cell-surface tumor antigens or taken up by the sample tumor cells;
(c) administering to the subject a diagnostically effective amount of at least one biologically compatible fluorescing targeting construct containing a compound determined in (b) that is tagged with a chemotherapeutic molecule to bind to and/or be taken up by exposed tumor tissue or organ that matches the sample tumor cells, which targeting construct fluoresces in response to light having at least one excitation wavelength in the range from 401 nm to 510 nm;
(d) diagnosing the location and/or surface area of the exposed tumor tissue in the in vivo body cavity or organ by directly viewing with or without the aid of an endoscopic device for viewing the fluorescence emanating from the targeting construct bound to or taken up in the exposed tumor tissue or organ in response to illumination with the at least one excitation light source; and thereafter
(e) leaving at least some of the targeting construct in contact with the tumor tissue for a sufficient time to kill cells of the tumor tissue.
